(19) United States
(12) Patent Application Publication
(10) Pub. No.: US 2026/0060983 A1
(43) Pub. Date: Mar. 5, 2026

(54) SUSTAINED RELEASE DELIVERY OF A PDE10 INHIBITOR

(71) Applicant: Merck Sharp & Dohme LLC, Rahway, NJ (US)

(72) Inventors: Seth P. Forster, Fort Washington, PA (US); Brian P. Regler, Aberdeen, NJ (US)

(73) Assignee: Merck Sharp & Dohme LLC, Rahway, NJ (US)

(21) Appl. No.: 19/104,450

(22) PCT Filed: Aug. 28, 2023
(86) PCT No.: PCT/US2023/031211

§ 371 Date: Feb. 18, 2025

Related U.S. Application Data
(60) Provisional application No. 63/402,625, filed on Aug. 31, 2022.

(51) Int. Cl. A61K 31/506; A61K 9/20; A61K 9/48

(52) U.S. Cl. CPC A61K 31/506; A61K 9/2013; A61K 9/2031; A61K 9/2095; A61K 9/4816; A61K 9/4833

(57) ABSTRACT

The invention relates to a sustained release pharmaceutical composition comprising a tablet core comprising a therapeutically effective amount of a compound selected from Group 1, or a pharmaceutically acceptable salt thereof, an insoluble matrix forming release agent and a hydrophilic matrix forming release agent.

### BACKGROUND OF THE INVENTION

The invention relates to compositions which provide sustained release of a PDE10 inhibitor.

Therapeutic compounds are often released quickly in the gastrointestinal tract, leading to sharp increases and subsequent relatively significant decreases in plasma level concentrations. The “Cₘₐₓ/Cₜᵢₘₑ” plasma concentration ratio is a value, at a specified time, calculated by dividing the maximum plasma level concentration by the plasma level concentration at the specific time. The value is useful for representing the speed with which a compound, administered to a patient, dissipates from the biological system. Compounds with high Cₘₐₓ/Cₜᵢₘₑ ratios may not provide the needed sustained, safe and efficacious therapeutic benefit. Such delivery poses potential safety concerns for certain drugs that are not well-tolerated at high levels.

Sustained release formulations have been prepared in a number of ways, generally to protect the active ingredient from exposure to stomach and intestine contents prior to the desired time of release. The compositions of the present invention solve the difficult problem of providing therapeutically safe and effective sustained plasma level concentration of a PDE10 inhibitor by controlling release of the PDE10 inhibitor through improvement of the granulation of the system and prolonging the drug release profile.

### SUMMARY OF THE INVENTION

The invention is a sustained release matrix pharmaceutical composition comprising a therapeutically effective amount of a compound selected from Group 1:
2-Methyl-6-{2-(5-methylpyridin-2-yl)cyclopropylimethoxyl-N-[(5-methyl-1,3,4-thiadiazol-2-yl)methyllpyrimidin-4-amine, 2-Methyl-6-{[(1 S,2S)-2-(5-methylpyridin-2-yl)cyclopropylimethoxyl-N-[(5-methyl-1,3,4-thiadiazol-2-yl)methyl]pyrimidin-4-amine,
S,S-2-methyl-N-[(5-methyl-1,3,4-thiadiazol-2-yl)methyl]-6-{[2-(1,5-naphthyridin-2-yl)cyclopropyl]methoxy}pyrimidin-4-amine, 6-{[(1 S,2S)-2-(5-methoxypyridin-2-yl)cyclopropyl]methoxy}-N-4-[(5-methyl-1,3,4-thiadiazol-2-yl)methyl]pyrimidine-2,4-diamine, 5-fluoro-6-{[(1 S,2S)-2-(5-methoxypyridin-2-yl)cyclopropyl]methoxy}-N-4-[(5-methyl-1,3,4-thiadiazol-2-yl)methyl]pyrimidine-2,4-diamine, 6-{[2-(3,3′-bipyridin-6-yl)cyclopropyl]methoxy}-2-methyl-N-[(5-methyl-1,3,4-thiadiazol-2-yl)methyl]pyrimidin-4-amine, 6-{[2-(5-cyclopropylpyridin-2-yl)cyclopropyl]methoxyl-2-methyl-N-[(5-methyl-1,3,4-thiadiazol-2-yl)methyl]pyrimidin-4-amine, and 2-amino-4-{[(1 S,2S)-2-(5-methylpyridin-2-yl)cyclopropyl]methoxy}-6-{[(5-methyl-1,3,4-thiadiazol-2-yl)methyl]aminolpyrimidine-5-carbonitrile, or a pharmaceutically acceptable salt thereof, a water insoluble matrix forming release agent, and a hydrophilic matrix forming release agent.

The invention is further related to a melt granulation sustained release matrix pharmaceutical composition comprising a therapeutically effective amount of a compound from Group 1, or a pharmaceutically acceptable salt thereof, water insoluble matrix forming release agent, and hydrophilic matrix forming release agent.

An embodiment of this invention is realized when the water insoluble matrix forming release agent is a modified glyceryl. A subembodiment of this aspect of the invention is realized when the modified glyceryl is selected from the group consisting of glycerol behenate (e.g., Compritol 888 ATO-sourced from Gattefossé), glyceryl palmitostearate (e.g., Precirol ATO 5), and glyceryl Tristearate (e.g., Dynasan 118).

Another embodiment of this invention is realized when the hydrophilic matrix forming release agent is selected from the group consisting of hydroxypropyl cellulose (HPC), hypromellose (HPMC) and polyethylene oxide (PEO). A subembodiment of this aspect of the invention is realized when the hydrophilic matrix forming release agent is HPC. Another subembodiment of this aspect of the invention is realized when the hydrophilic matrix forming release agent is HPMC. Still another subembodiment of this aspect of the invention is realized when the hydrophilic maxtrix forming release agent is PEO. Yet another subembodiment of this aspect of the invention is realized when the PEO is nonionic. A further subembodiment of this aspect of the invention is realized when the PEO has a mean molecular weight (g/mol) range of 100,000 to 10,000,000; 1,000,000 to 10,000,000; 4,000,000-10,000,000; 5,000,000 to 8,000,000; or 6,000,000 to 7,000,000; preferably 7,000,000 g/mol.

An embodiment of this invention is realized when the sustained release matrix pharmaceutical composition of this invention is a mixture of a compound selected from Group 1, or a pharmaceutically acceptable salt thereof and 1:1, 2:1, 2:3, 1:2, 3:1, 1:3 or 1:4 ratio of water insoluble matrix forming release agent, and hydrophilic matrix forming release agent, respectively. A subembodiment of this aspect of the invention is realized when the ratio of water insoluble matrix forming release agent, and hydrophilic matrix forming release agent is 1:1, respectively. A subembodiment of this aspect of the invention is realized when the ratio of water insoluble matrix forming release agent, and hydrophilic matrix forming release agent is 2:1, respectively. A subembodiment of this aspect of the invention is realized when the ratio of water insoluble matrix forming release agent, and hydrophilic matrix forming release agent is 2:3, respectively. Another subembodiment of this aspect of the invention is realized when the ratio of water insoluble matrix forming release agent, and hydrophilic matrix forming release agent is 1:2, respectively. Another subembodiment of this aspect of the invention is realized when athe ratio of water insoluble matrix forming release agent, and hydrophilic matrix forming release agent is 3:1, respectively. Still another subembodiment of this aspect of the invention is realized when the ratio of water insoluble matrix forming release agent, and hydrophilic matrix forming release agent is 1:3, respectively. Yet another subembodiment of this aspect of the invention is realized when the ratio of water insoluble matrix forming release agent, and hydrophilic matrix forming release agent is 1:4, respectively.

Another embodiment of this invention is realized when the sustained release matrix pharmaceutical composition of this invention is a mixture prepared by melt granulation of a compound selected from Group 1, or a pharmaceutically acceptable salt thereof and 1:1, 1:2, 1:3 or 1:4 ratio of water insoluble matrix forming release agent selected from the group consisting of glycerol behenate (e.g., Compritol 888 ATO), glyceryl palmitostearate (e.g., Precirol ATO 5), and glyceryl Tristearate (e.g., Dynasan 118), and hydrophilic matrix forming release agent, respectively.

Another embodiment of this invention is realized when the sustained release matrix pharmaceutical composition of this invention is a mixture prepared by melt granulation of a compound selected from Group 1, or a pharmaceutically acceptable salt thereof and 1:1, 1:2, 1:3 or 1:4 ratio of water insoluble matrix forming release agent and hydrophilic matrix forming release agent selected from hydroxypropyl cellulose (HPC), Hypromellose (HPMC) and polyethylene oxide (PEO), respectively. A subembodiment of this aspect of the invention is realized when the hydrophilic matrix forming release agent is HPC. Another subembodiment of this aspect of the invention is realized when the hydrophilic matrix forming release agent is HPMC. Still another subembodiment of this aspect of the invention is realized when the hydrophilic maxtrix forming release agent is PEO. Yet another subembodiment of this aspect of the invention is realized when the PEO is nonionic. A further subembodiment of this aspect of the invention is realized when the PEO has a mean molecular weight range of 100,000 to 10,000,000; 1,000,000 to 10,000,000; 4,000,000-10,000,000; 5,000,000 to 8,000,000; or 6,000,000 to 7,000,000; preferably 7,000,000.

Another embodiment of this invention is realized when the sustained release matrix pharmaceutical composition of this invention is a mixture prepared by melt granulation of a compound selected from Group 1, or a pharmaceutically acceptable salt thereof and 1:1, 1:2, 1:3 or 1:4 ratio of water insoluble matrix forming release agent and hydrophilic matrix forming release agent polyethylene oxide (PEO), respectively, wherein the PEO has a mean molecular weight range selected from 4,000,000-10,000,000; 5,000,000 to 8,000,000; and 6,000,000 to 7,000,000. A subembodiment of this aspect of the invention is realized when the mean molecular weight range is 7,000,000.

Another embodiment of this invention is realized when the sustained release matrix pharmaceutical composition of this invention is a mixture prepared by melt granulation of a compound selected from Group 1, or a pharmaceutically acceptable salt thereof and 1:1 ratio of water insoluble matrix forming release agent, glycerol behenate, and hydrophilic matrix forming release agent PEO, respectively, wherein the PEO has a mean molecular weight range of 4,000,000-10,000,000; 5,000,000 to 8,000,000; or 6,000,000 to 7,000,000. A subembodiment of this aspect of the invention is realized when the mean molecular weight range of PEO is 7,000,000.

Another embodiment of this invention is realized when the sustained release matrix pharmaceutical composition of this invention is a mixture prepared by melt granulation of a compound selected from Group 1, or a pharmaceutically acceptable salt thereof, and 1:2 ratio of water insoluble matrix forming release agent, glycerol behenate, and hydrophilic matrix forming release agent PEO, respectively, wherein the PEO has a mean molecular weight range of 4,000,000-10,000,000; 5,000,000 to 8,000,000; or 6,000,000 to 7,000,000. A subembodiment of this aspect of the invention is realized when the mean molecular weight range of PEO is 7,000,000.

Still another embodiment of this invention is a tablet core comprising a therapeutically effective amount of a compound selected from Group 1, or a pharmaceutically acceptable salt thereof, a water insoluble matrix forming release agent and hydrophilic matrix forming release agent. A subembodiment of this aspect of the invention is realize when the tablet core is optionally coated coated.

Another embodiment of the present invention is realized when a neutralizing agent is used in the sustained release matrix pharmaceutical composition formulation.

An embodiment of this invention is realized by a sustained release matrix pharmaceutical melt granulated formulation of 20% or less by weight of a compound selected from Group 1, or a pharmaceutically acceptable salt thereof, in a 1:1, 1:2, or 1:3 ratio of water insoluble matrix forming release agent and hydrophilic matrix forming release agent, respectively. A subembodiment of this invention is realized by a sustained release matrix pharmaceutical melt granulated formulation of 0.5% to 20%, 2%-10%, 2%-6%, 4%-5%, preferably 5% by weight of a compound selected from Group 1, or a pharmaceutically acceptable salt thereof, in a 1:1, 1:2, or 1:3 ratio of water insoluble matrix forming release agent and hydrophilic matrix forming release agent, respectively.

In another embodiment of compositions, the tablet core comprises an amount of a compound selected from Group 1, or a pharmaceutically acceptable salt thereof, of between 1% and 10% by weight of the total core mass, and about 90%-99%, preferably about 80%-98, of a 1:1, 1:2, or 1:3 ratio amount of a water insoluble matrix forming release agent and hydrophilic matrix forming release agent, respectively, by weight of the total core mass. As aspect of this embodiment is realized when optional tablet excipients are added. A further aspect of this embodiment is realized when 0.5%-20% w/w of optional tablet excipients are added.

In another embodiment of compositions, the tablet core comprises an amount of a compound selected from Group 1, or a pharmaceutically acceptable salt thereof, of between 1% and 10% by weight of the total core mass and Glyceryl Behenate and Polyethylene Oxide weight percentages of about 49.5% and 49.5%, 24.75% and 74.25%, 74.25% and 24.75%, 40% and 40%, 20% and 60%, 60% and 20%, respectively. A subembodiment of this aspect of the invention is realized when the Glyceryl Behenate and Polyethylene Oxide weight percentages is about 49.5% and 49.5%, respectively. Another subembodiment of this aspect of the invention is realized when the Glyceryl Behenate and Polyethylene Oxide weight percentages is about 24.75% and 74.25%, respectively. Another subembodiment of this aspect of the invention is realized when the Glyceryl Behenate and Polyethylene Oxide weight percentages is about 74.25% and 24.75%, respectively. Another subembodiment of this aspect of the invention is realized when the Glyceryl Behenate and Polyethylene Oxide weight percentages is about 40% and 40%, respectively. Another subembodiment of this aspect of the invention is realized when the Glyceryl Behenate and Polyethylene Oxide weight percentages is about 20% and 60%, respectively. Another subembodiment of this aspect of the invention is realized when the Glyceryl Behenate and Polyethylene Oxide weight percentages is about 60% and 20%, respectively. An embodiment of this aspect of the invention is realized when the tablet core is compressed.

The invention also comprises a method for orally administering, to a patient, a therapeutically effective dose of the active ingredient in a pharmaceutical composition core comprising a compound selected from Group 1, or a pharmaceutically acceptable salt thereof, in a 1:1 a water insoluble matrix forming release agent and hydrophilic matrix forming release agent, respectively, and an optional neutralizing agent (5%-20% w/w).

Compositions of the invention are useful in the manufacture of a medicament for sustained release of the active ingredient, a compound selected from Group 1.

### BRIEF DESCRIPTION OF THE DRAWING

FIG. 1 shows that active ingredient plasma level concentration over time was maintained at a moderate level, with decreased peak concentration, compared to immediate release tablets.

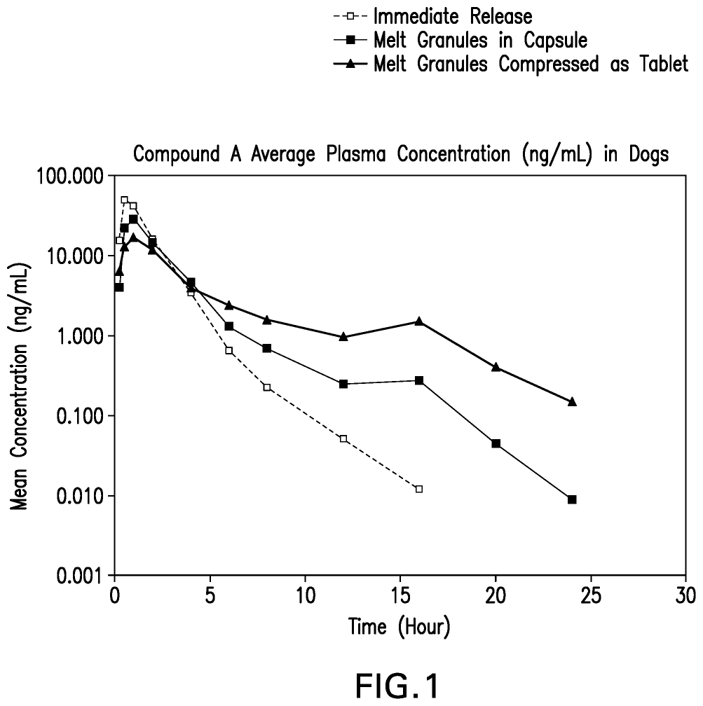

### DETAILED DESCRIPTION OF THE INVENTION

By “pharmaceutically active agent,” “active ingredient,” “medicament,” or “beneficial agent” is meant a compound selected from Group 1, and pharmaceutically acceptable salts thereof, and derivatives that produce similar localized or systemic effect or effects in animals. Derivatives of the active ingredient, such as esters, ethers and amides without regard to their ionization and solubility characteristics can be used alone or mixed with other compounds. Also, prodrugs of the active agent can be used in a form that, upon release from the tablet, is converted by enzymes, hydrolyzed by body pH or converted by other metabolic processes, to the original form, or to a biologically active form. That is, prodrugs are specifically included within the definition of pharmaceutically active ingredients. Some of the active ingredients included within the compositions of the present invention are chiral; included within the scope of the present invention compositions are those having racemic mixtures and separated enantiomers of the active ingredient. Furthermore, hydrates as well as anhydrous compositions and polymorphs of the active ingredient may be included in compositions of the present invention.

The term “pharmaceutically acceptable salts” means non-toxic salts of the active ingredients which are generally prepared by reacting the free base with a suitable organic or inorganic acid. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylbromide, methylnitrate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, valerate.

The term “hydrophilic matrix forming release agent”, also referred to as “water swellable polymer”, and all refer to a polymer used in the tablet core that is capable of swelling upon hydration. The polymer is capable of producing discrete gel particles which support a suspension, including the active ingredient. The term “swellable” implies that the polymer is in a non-hydrated state, while the term “swelling” implies that the polymer is in a hydrated state. The water swellable polymer used also must exude from the core surface in such a way that the beneficial agent is carried into the environment of use. Upon hydration, the gel particles must be predisposed to leave the surface taking the drug with it. This assures a constant surface area exposed to the solvent of the environment of use and maintains the appropriate rate of release. The gel particles are composed of discrete particles of hydrated polymer. Both size and hydration rate of these microscopic gel particles are characteristics of the hydrated polymer. Illustrative of swellable polymer which forms gel particles are polyethylene oxide (PEO). Examples of PEOs useful in the claimed invention are Dow: POLYOX WSR-303 NF, WSR Coagulant NF, WSR-301 NF; Sumitomo Seika Chemicals Co., LTD: PEO-29, PEO-27, PEO-18; and the like.

Other non-limiting swellable polymers (hydrophilic matrix forming release agents) useful in the invention are hypromellose (HPMC), hydroxypropyl cellulose (HPC), “AQUAKEEP J-550”, “AQUAKEEP J-400”, which are trade names for sodium acrylate polymer produced by Seitetsu Kagaku Co., Ltd., Hyogo, Japan. The “AQUAKEEP” polymers are generically described in U.S. Pat. No. 4,340,706. Also illustrative of this type of polymer are the carboxypolymethylenes prepared from acrylic acid cross-linked with allyl ethers of sucrose or pentaerythritol and sold under the trade names “CARBOPOL 934P” and “CARBOPOL 974P” which are trade names for two carbamer type polymers produced by B.F. Goodrich Chemical Company, Cleveland, Ohio. Carbamer polymers are generically described in U.S. Pat. No. 2,909,462 and in the National Formulary XVII at p. 1911, CAS Registry Number 9003-01-4. See also U.S. Pat. No. 6,635,280. All of the forgoing references are hereby incorporated by reference. In the dry state, “CARBOPOL 974P” and “CARBOPOL 934P” particles range in size from 2 to 7 microns. When these dry particles are hydrated, microscopic gel particles in the range of 20 microns are produced. When “AQUAKEEP J-550” or “AQUAKEEP J-400” dry particles are hydrated, microscopic gel particles diameter can range in size from 100 to 1000 microns.

The term “water insoluble matrix forming release agent” is defined as an agent comprising modified glycerol water insoluble lipid excipients. Suitable modified glycerol release agents include glyceryl palmitostearate (e.g. Precirol ATO 5), glyceryl tristearate (e.g., Dynasan 118), and glyceryl behenate (e.g., Compritol 888 ATO).

In an aspect of this invention Glyceryl behenate (GB) is operating as a water insoluble matrix former and PEO is operating as a hydrophilic matrix former. The combination of the two together as matrix formers appear to slow the gelling of the system overall and extend the duration of release. In another aspect of this invention the hydrophobic GB coats the PEO, slowing its hydration, gelling, and dissolution. An embodiment of this invention is realized by a sustained release matrix pharmaceutical melt granulated formulation of 0.5% to 20%, 2%-10%, 2%-6%, 4%-5%, preferably 5% a compound selected from Group 1, or a pharmaceutically acceptable salt thereof, in a 1:1, 1:2, or 1:3 ratio, respectively, of GB and hydrophilic matrix forming release agent. An embodiment of this aspect of the invention is realized when there is 20%-60% GB and 35-75% of hydrophilic matrix forming release agent.

When used, the “neutralizing agent” modulates swellable polymer hydration and provides for release of the active ingredient from the tablet core into the gastrointestinal tract by diffusion directly from the core and by extrusion of swelling polymer. The neutralizing agent is solubilized by the aqueous media of the environment and establishes an environment such that the environment pH, ionic strength or hydrophilic character is appropriate for the desired polymer gel particle hydration rate. For example, the neutralizing agents can enhance or retard the neutralization of acidic functional groups on the polymer, thereby affecting the hydration rate. Among the suitable neutralizing agents for use in the invention are bases and the salts of bases such as sodium carbonate, sodium bicarbonate, betaine hydrochloride, sodium citrate, arginine, meglamine, sodium acetate, sodium phosphates (e.g., sodium phosphate dibasic anhydrous), potassium phosphates, calcium phosphate, ammonium phosphate, magnesium oxide, magnesium hydroxide, sodium tartrate and tromethamine. Other compounds that can be used as neutralizing agents include sugars such as lactose, sucrose, mannitol, sorbitol, pentaerythritol, glucose and dextrose. Polymers such as microcrystalline cellulose and polyethylene glycol, as well as surfactants and other organic and inorganic salts can also be used to modulate polymer hydration. Most preferably, sodium phosphate dibasic anhydrous is used.

By “drug delivery device” is meant, a dosage form that provides a convenient means of delivering a pharmaceutically active ingredient or drug to a subject in need thereof. The subject can be a human or any other animal in need of such pharmaceutically active ingredient. The device is designed to be useful for the delivery of a pharmaceutically active ingredient by any pharmaceutically accepted means such as by swallowing, retaining it within the mouth until the beneficial agent has been dispensed, placing it within the buccal cavity, or the like.

By “sustained” is meant that the rate of release of the pharmaceutically active ingredient from the device to the environment of use is not immediate, but rather, follows a predetermined pattern. Thus, relatively constant or predictably varying amounts of the beneficial agent can be dispensed over a specified period of time.

The term “animal” includes mammals, humans and primates such as domestic, household, sport or farm animals such as sheep, goats, cattle, horses and pigs, laboratory animals such as mice, rats and guinea pigs, fishes, avians, reptiles and zoo animals.

An aspect of the instant invention is realized when the composition is in the form of a compressed core. By “compressed core” is meant composition of the invention that is blended in such a way to produce a uniform material that is compressed, within a die, to produce a desired form, normally in the shape of a tablet, capsule or bolus.

The compressed core contains a “therapeutically effective amount” of active ingredient. By “therapeutically effective amount” is meant that the quantity of pharmaceutically active ingredient which has been demonstrated to be sufficient to induce the desired effect during studies utilizing the compound.

The formed gel particles are a system consisting of hydrated polymer interpenetrated by the aqueous solvent of the environment of use.

### Tablet Core

In one embodiment of the tablet core, the amount of active ingredient is between about 1% and about 20% by weight of the total core mass (e.g., between 5 mg and 50 mg or more, e.g. 5 mg, 10 mg, 20 mg, 25 mg, 30 mg, 40 mg, 50 mg), and the amount of water insoluble matrix forming release agent and hydrophilic matrix forming release agent mixture is between about 80% and about 99% by weight of the total core mass. An embodiment of this aspect of the invention is realized when the ratio of water insoluble matrix forming release agent and hydrophilic matrix forming release agent, respectively, is selected from 1:1, 1:2, 1:3. Another embodiment of this aspect of the invention is realized when the ratio is 1:1. Another embodiment of this aspect of the invention is realized when the tablet weights range from 20-400 mg.

The tablet core is typically in the form of a solid conventional tablet. Generally, the core is compressed into its final shape using a standard tablet compressing machine. The core may contain compressing aids and diluents such as lactose that assist in the production of compressed tablets. The core can be comprised of a mixture of agents combined to give the desired manufacturing and delivery characteristics. The number of agents combined to make the core is substantially without an upper limit, and with a lower limit of two agents: the water swellable polymer, and the active ingredient. Optionally, a third agent, which is a neutralizing agent, can be used.

The active ingredient may be in the core as a dispersion, particle, granule, or powder. Also, the pharmaceutically active ingredient may be mixed with a binder, dispersant, emulsifier or wetting agent and dyes. Other excipients such as lactose (e.g. Lactose NF hydrous spray dry), magnesium stearate, microcrystalline cellulose (e.g. Avicel PH 101), starch, stearic acid, calcium phosphate, glycerol monostearate, sucrose; polyvinylpyrrolidone, gelatin, methylcellulose, sodium carboxymethylcellulose, sorbitol, mannitol, polyethylene glycol and other ingredients commonly utilized as stabilizing agents or to aid in the production of tablets may also be present in the core.

In instances where the pharmaceutically active ingredient, and the water insoluble matrix forming release agent and hydrophilic matrix forming release agent mixture exhibit the desired release rate, stability, and manufacturing characteristics, the ratio of drug to excipient is dictated by the desired time span and profile of release, and the pharmacological activity of the drug.

Once the core is prepared, it may be coated by a coating such as a water soluble coating, or a coating that enables enteric release (pH dependent solubility).

Once the composition is within the environment of use, the polymer of the compressed core which is exposed to the ambient aqueous solution at the tablet surface begins to hydrate and produce gel particles. During the production of the swelling polymer, soluble and insoluble core components located near the polymer particles become dispersed and mixed in such a manner that a gelatinous dispersion is produced. The dispersion moves a portion of the active ingredient from the core into the aqueous solvent, bringing the beneficial agent into the environment of use. Another portion of active ingredient move into the environment free of interaction with the swelling polymer.

In operation, aqueous solution from the environment of use (e.g., gastrointestinal system) contacts the surface of the core that is exposed. The available water begins to hydrate the polymer at the surface of the core. The neutralizing agent, if employed, is present at the exposed core surface, and is solubilized and may optionally be used to adjust local pH or polymer hydration. As the polymer particles are hydrated, the swelling polymer moves from the surface. At the same time, the swelling polymer moves a portion of the active ingredient from the surrounding surface by extrusion into the environment. The swelling polymer moves from the core surface into the environment of use in a dispersion. Another portion of the active ingredient moves from the core into the environment by diffusion. As a result, controlling the surface area of the core which is exposed to the environment of use, the portion of active ingredient moved into the environment by the swelling polymer, and the portion of active ingredient diffused directly into the environment, effectively controls the delivery rate of medicament to the environment. The neutralizing agent regulates gel formation at a rate which allows for a portion of the highly water-soluble active ingredient to become integrated within the gel matrix formed by the polymer, and allows for another portion to freely move from the core directly into the aqueous environment. Delivery of the active ingredient occurs from the surface of the core and from within the core so that the delivery rate is dependent on diffusion of the active ingredient from inside the core to the environment of use as well as extrusion of the swelling polymer, containing the active ingredient, from the core.

The following examples illustrate the preparation of the drug delivery device of this invention and the sustained release of active ingredient into an environment of use, and are not to be considered as limiting the invention set forth in the claims appended hereto.

The dosage regimen utilizing the compositions of the present invention is selected in accordance with a variety of factors including type, species, age, weight, sex and medical condition of the patient; the severity of the condition to be treated; the route of administration; the renal and hepatic function of the patient; and the particular active ingredient or salt thereof employed. An ordinarily skilled physician or veterinarian can readily determine and prescribe the effective amount of the drug required to prevent, counter, or arrest the progress of the condition.

Representative compositions of the instant invention include those that comprise, for example, 1-20% Compound A, 10%-75% polyethylene oxide, 1 MDA and 10%-75% glyceryl behenate for a total of 100%; or 1-20% Compound A, 10%-75% polyethylene oxide, 100 kDa, and 10%-75% glyceryl behenate for a total of 100%.

In the following examples, the Group I compound 2-Methyl-6-{[(1S,2S)-2-(5-methylpyridin-2-yl)cyclopropyl]methoxy}-N-[(5-methyl-1,3,4-thiadiazol-2-yl)methyl]pyrimidin-4-amine (Compound A) is the active ingredient.

### Example

Compositions containing 1-50 mg, respectively, of Compound A and 1:1, 1:3, or 3:1 ratio of GB and PEO, respectively were prepared using the following ingredients and amounts and formulation procedures.

| Tablet Core Ingredients | |
|---|---|
| Example 1 | % (weight) |
| Compound A | 5% |
| Polyethylene Oxide, 7 MDa | 47.5% |
| Glyceryl Behenate | 47.5% |
| | 100% |

Tablet Core Ingredients
Example 2

| | |
|---|---|
| Compound A | 20% |
| Polyethylene Oxide, 7 MDa | 40% |
| Glyceryl Behenate | 40% |
| | 100% |

Tablet Core Ingredients
Example 3

| | |
|---|---|
| Compound A | 5% |
| Polyethylene Oxide, 7 MDa | 23.75% |
| Glyceryl Behenate | 71.25% |
| | 100% |

Tablet Core Ingredients
Example 4

| | |
|---|---|
| Compound A | 5% |
| Polyethylene Oxide, 7 MDa | 71.25% |
| Glyceryl Behenate | 23.75% |
| | 100% |

### Example 5

Each of the formulations of Examples 1, 2, 3 and 4 were co-granulated using a twin-screw extruder or high-shear wet granulator. The granules were then milled using a cone mill to produce consistent granule particle size. The granules were then put into capsules, or compressed into tablets at compaction pressures of 100-400 MPa.

Total weight of finished tablets as a function of amount of active ingredient is exemplified as follows:

| | |
|---|---|
| Active ingredient | Total tablet weight |

| | | | |
|---|---|---|---|
| 4 | mg | 80 | mg |
| 12 | mg | 240 | mg |
| 20 | mg | 400 | mg |

Several formulations were orally administered to dogs. Table 2 compares the summary pharmacokinetic (PK) parameters for the formulations, and shows that the active ingredient as formulated in Example 1 was released in a sustained manner for both sustained release capsules (CR4) and sustained release tablets (CR5) compared to immediate release (IR) capsules.

TABLE 1
IR capsule formulation:

| | Unit formula |
|---|---|
| Material Description | (mg) |
| Compound A | 2 |
| Calcium Phosphate, Dibasic, Anhydrous | 113.9 |
| Cellulose, Microcrystalline | 56.93 |
| Croscarmellose Sodium | 5.4 |
| Magnesium Stearate | 1.8 |
| Total: | 180 |

TABLE 2

| | | | | | | Bioavailability |
|---|---|---|---|---|---|---|
| | AUC, 0-24 hr | AUC, 0-Inf | Cmax | Tmax | | Relative to IR |
| Treatment | [nmol/L *hr] | [nmol/L * hr] | [nmol/L] | [h] | | [%] |
| IR Capsule | 229 ± 32 | 229 ± 33 | 144.7 ± 13.4 | 0.5 | (0.5-1) | 100 |
| SR Capsule | 180 ± 39 | 181 ± 39 | 87.9 ± 23.2 | 1 | (0.5-1) | 79 |
| (CR4) | | | | | | |
| SR Tablet | 173 ± 114 | 175 ± 116 | 51.3 ± 30.0 | 0.75 | (0.25-1) | 76 |
| (CR5) | | | | | | |

### Table 2: Summary PK Parameters, CR4=Melt Granules in Capsule, CR5=Melt Granules Compressed as Tablets

1. A sustained release pharmaceutical composition comprising a tablet core comprising a therapeutically effective amount of
a) a compound selected from Group 1:
2-Methyl-6-{2-(5-methylpyridin-2-yl)cyclopropylimethoxyl-N-[(5-methyl-1,3,4-thiadiazol-2-yl)methyllpyrimidin-4-amine,
2-Methyl-6-{[(1 S,2S)-2-(5-methylpyridin-2-yl)cyclopropylimethoxyl-N-[(5-methyl-1,3,4-thiadiazol-2-yl)methyl]pyrimidin-4-amine,
S,S-2-methyl-N-[(5-methyl-1,3,4-thiadiazol-2-yl)methyl]-6-{[2-(1,5-naphthyridin-2-yl)cyclopropyl]methoxy}pyrimidin-4-amine,
6-{[(1 S,2S)-2-(5-methoxypyridin-2-yl)cyclopropyl]methoxy}-N-4-[(5-methyl-1,3,4-thiadiazol-2-yl)methyl]pyrimidine-2,4-diamine,
5-fluoro-6-{[(1 S,2S)-2-(5-methoxypyridin-2-yl)cyclopropyl]methoxy}-N-4-[(5-methyl-1,3,4-thiadiazol-2-yl)methyl]pyrimidine-2,4-diamine,
6-{[2-(3,3′-bipyridin-6-yl)cyclopropyl]methoxy}-2-methyl-N-[(5-methyl-1,3,4-thiadiazol-2-yl)methyl]pyrimidin-4-amine,
6-{[2-(5-cyclopropylpyridin-2-yl)cyclopropyl]methoxyl-2-methyl-N-[(5-methyl-1,3,4-thiadiazol-2-yl)methyl]pyrimidin-4-amine, and
2-amino-4-{[(1 S,2S)-2-(5-methylpyridin-2-yl)cyclopropyl]methoxy}-6-{[(5-methyl-1,3,4-thiadiazol-2-yl)methyl]aminolpyrimidine-5-carbonitrile, or a pharmaceutically acceptable salt thereof,
b) a water insoluble matrix forming release agent, and
c) a hydrophilic matrix forming release agent.

2. The composition of claim 1, wherein the tablet core comprises an amount of a compound from Group 1, or a pharmaceutically acceptable salt thereof between about 1% and about 10% by weight of the total core mass, and between 90% and about 99% of a 1:1, 2:1, 2:3, 1:2, 3:1, 1:3 or 1:4 ratio amount of a water insoluble matrix forming release agent and hydrophilic matrix forming release agent, respectively, by weight of the total core mass.

3. The composition according to claim 1, which is formed by melt granulation.

4. The composition according to claim 1, wherein the water insoluble matrix forming release agent is a modified glyceryl.

5. The composition according to claim 4, wherein the modified glyceryl is selected from the group consisting of glycerol behenate, glyceryl palmitostearate, and glyceryl tristearate.

6. The composition according to claim 1, wherein the hydrophilic matrix forming release agent is selected from the group consisting of hydroxypropyl cellulose (HPC), Hypromellose (HPMC) and polyethylene oxide (PEO).

7. The composition according to claim 6 wherein the hydrophilic matrix forming release agent is PEO.

8. The composition according to claim 7 wherein the PEO has a mean molecular weight range of 4,000,000 to 10,000,000.

9. The composition according to claim 1, wherein the sustained release matrix pharmaceutical composition is prepared by melt granulation of a compound selected from Group 1, or a pharmaceutically acceptable salt thereof and 1:1, 1:2, 1:3 or 1:4 ratio of a water insoluble matrix forming release agent selected from the group consisting of glycerol behenate, glyceryl palmitostearate, and glyceryl tristearate, and a hydrophilic matrix forming release agent, respectively.

10. The composition according to claim 9 wherein the hydrophilic matrix forming release agent is PEO.

11. The composition according to claim 10 wherein hydrophilic matrix forming release agent is PEO having a mean molecular weight range of 5,000,000 to 8,000,000.

12. The composition according to claim 1, wherein the sustained release matrix pharmaceutical composition is prepared by melt granulation of a compound selected from Group 1, or a pharmaceutically acceptable salt thereof and 1:1 ratio of water insoluble matrix forming release agent and hydrophilic matrix forming release agent, respectively.

13. The composition according to claim 12 wherein the insoluble matrix forming release agent is selected from the group consisting of glycerol behenate, glyceryl palmitostearate, and glyceryl tristearate, and the hydrophilic matrix forming release agent is PEO having a mean molecular weight range of 5,000,000 to 8,000,000.

14. The composition according to claim 12, wherein the insoluble matrix forming release agent is glycerol behenate and the hydrophilic matrix forming release agent is PEO.

15. The composition according to claim 1, wherein the composition comprises a compressed core.

16. The composition according to claim 1, further comprising a neutralizing agent.
